(19) United States
(12) Patent Application Publication
(10) Pub. No.: US 2007/0014757 A1
(43) Pub. Date: Jan. 18, 2007

(54) Compositions and complexes containing a macromolecular compound as potential anti-inflammatory agents

(75) Inventors: Abhay Singh Chauhan, Hyderabad (IN); Prakash Vamanrao Diwan, Hyderabad (IN); Narendra Kumar Jain, Sagar (IN); Kondapuram Vijaya Raghavan, A.P. (IN)

Correspondence Address: FOLEY AND LARDNER LLP;SUITE 500, 3000 K STREET NW, WASHINGTON, DC, 20007, US

(73) Assignee: COUNCIL OF SCIENTIFIC AND INDUSTRIAL RESEARCH

(21) Appl. No.: 11/524,885

(22) Filed: Sep. 22, 2006

Related U.S. Application Data
(63) Continuation of application No. 10/103,137, filed on Mar. 22, 2002.

(51) Int. Cl. A61K 31/787; A61K 31/785

(52) U.S. Cl. 424/78.27; 977/754

(57) ABSTRACT

A composition exhibiting anti-inflammatory activity comprising of a momodisperse macromolecular polymers such as dendrimer having a plurality of terminal groups or such molecules bound/complexed to drug moieties having anti-inflammatory activity or which assist in anti-inflammatory activity and its use in the pharmaceutical formulation for treating disease or pathological conditions associated with inflammation.

### FIELD OF THE INVENTION

The present invention relates to pharmacologically-active, anti-inflammatory compound dendrimer/other monodisperse macromolecular polymer/their analogue or such molecules attached to drug having anti-inflammatory activity or which assist in anti-inflammatory activity, thus useful for treating diseases and pathological conditions involving inflammation The present invention pertains to pharmacological activity including, but not limited, to the use as an anti-inflammatory drug. Complexes of dendrimer with anti-inflammatory drug and compositions for anti-inflammatory activity are also disclosed.

### BACKGROUND OF THE INVENTION

Inflammation refers to a response to an tissue injury caused by pathogenic microorganisms, trauma, chemicals and heat in view of restoring the injured tissue, that is, the whole local tissue response to an injury involving secretion of several mediators from the injured tissue, induction of immunocytes and recovery of the injured tissue. This process can be summarized as follows. With tissue cells damaged or destroyed, acids and chemical mediators are released. The mediators cause the dilation of blood capillaries and increase their permeability. Histamine secreted from mast cells or basophiles initiates the response of blood vessels, and serum kinin produced from alpha-2-globulin of blood serum mediates the long-acting response of blood vessels through the blood coagulation mechanism. The blood capillary dilation increases the blood flow, and causes heat and redness. The increased permeability of the blood capillaries cause blood cells, proteins and fluids to exude into surrounding tissues, leading to swelling. Such exudation can accelerate further destruction of cells, and the increased blood pressure stimulates peripheral nerves to cause pain. The pain increases due to secretion of kinin and acids. Other mediators secreted from the tissue include serotonin, prostaglandin, reactants of the complement system, and lymphokine secreted from T-cells.

As fluid exudes from the capillaries, leukocytes (i.e., neutrophils and monocytes) migrate to the damaged region and digest or dissolve inflammation-causing substances to recover the damaged area. Another important cells in the inflammatory reaction are monocyte-originated macrophages that also participate in phagocytosis and rapidly proliferate when the tissue is damaged. Fusion of the macrophages or amitotic division of large fragments produces giant cells.

As described, inflammation is a primary mechanism of the body system to repair tissue damage or protect against latent infection. However, an untimely or chronic inflammation reaction can result in pain or disability.

Any inflammation that occurs in the mammalian body is the clinical result of a sequence of events known as the arachidonic acid cascade. Cell membranes consist of phospholipids. including fatty acids, one of which is ARA. In the inflammation process, the first step is the release of ARA from the phospholipid. The next step is the conversion of ARA into the specific mediator of inflammation. One pathway is the cyclooxygenase and the other is called the lipoxygenase pathway. Cortisone, along with other selected steroidal agents block both inflammation pathways by inhibiting ARA release.

There can be two types of anti-inflammatory medications, the one of which involves inhibiting production and exudation of inflammatory cells and the other involves reducing secretion of inflammation mediators. The currently used medical agents may be divided into NSAIDs, capable of producing both analgesic and inflammatory effects as described above, and steroidal anti-inflammatory drugs. The NSAIDs are widely spread as analgesic and inflammatory agents and have a mechanism of inhibiting production of prostaglandin from arachidonic acid. Corticosteroids used against inflammation not only inhibit generation of prostaglandin but also act on beta-adrenergic receptors of leukocytes to inhibit secretion of inter-leukins (ILs) and reduce permeability of the blood vessels, which in turn inhibits exudation of blood and inflammatory cells. Despite the therapeutic effects, corticosteroids have been reported to produce a number of side effects, such as increasing the size of erythrocytes, weight gain, accelerating progression of osteoporosis and weakening blood capillary, raising blood pressure and stomach ulcer.

The mechanism whereby an NSAID induces anti-inflammatory may be attributed to its known inhibition of cyclooxygenase-2 (COX-2), an enzyme associated with the inflammatory process. Prostaglandins are synthesized by the cyclooxygenase enzyme, of which there are two known isoforms, COX-1 and COX-2. COX-1 is a constitutive enzyme expressed in many tissues including the gastric mucosa, whereas COX-2 is an inducible enzyme expressed in fibroblasts, macrophages and other cell types in inflammation. Although NSAIDs can inhibit both COX isoforms, they are selective in their inhibition rates of these enzymes. It has been suggested that the GI side effects associated with NSAIDs relate to COX-1 inhibition, while the anti-inflammatory effects of NSAIDs, relate to COX-2 inhibition.

However, there are some problems associated with NSAIDs treatment including delivery to the appropriate site of action and side effects. A disadvantage of most NSAID therapy is that the NSAID is given systemically, and for long periods. Prolonged high systemic concentrations of many NSAIDs can result in other complications unrelated to the regular treatment. For example, such NSAID users have a three-fold greater risk of developing serious gastro-intestinal complications over non-NSAID users. It has been estimated that 20% to 40% of patients on systemic NSAID therapy develop peptic ulcers. It has also been estimated that 10,000 fatalities a year occur in the United States from NSAID-induced gastrointestinal disorders. Other adverse effects of NSAIDs include renal failure, hepatic dysfunction, and bleeding and gastric ulceration. The side effects of NSAIDs are especially of concern in the elderly. Therefore, a need exists for an alternative method to target therapeutic concentrations of NSAIDs.

Non-steroidal anti-inflammatory drugs (NSAIDS) have been effective in reducing inflammation and inducing analgesia; however, the conventional oral dosage forms of these drugs characteristically have short half-lives and irritate the gastrointestinal mucosa. Therefore, currently available slow release oral dosage forms of NSAIDs induce systemic effects and the drug is not efficiently used at the site of inflammation. Further, in the currently available slow release oral dosage forms of NSAIDs, fillers or additives are needed in order to accelerate or retard drug release. Further still, large doses of NSAIDs administered by conventional dosing regimens often times result in toxicity and secondary pathology such as gastrointestinal tissue irritation.

The side effects and draw back of anti-inflammatory therapy can be dealt either by invention of drugs without side effect or by targeting the presently available drugs to the specific site by using drug delivery systems.

The present invention provides a new class of anti-inflammatory agents based on a particular type of polymer referred to herein as a “dendrimer”, which have substantial inherent anti inflammatory activity, without causing any gastrointestinal complications and also can act as macromolecular drug delivery system for anti-inflammatory drugs with sustained action and better targeting could be achieved. These compounds are therefore well suited for prophylactic and therapeutic use as anti-inflammatory agents in humans and animals.

### SUMMARY OF THE INVENTION

Accordingly, the present invention provides a composition exhibiting anti-inflammatory activity comprising of a momodisperse macromolecular compound such as dendrimer having a plurality of terminal groups or such molecules bound/complexed to drug moieties having anti-inflammatory activity or which assist in anti-inflammatory activity and its use in the pharmaceutical formulation for treating disease or pathological conditions associated with inflammation.

### DETAILED DESCRIPTION OF THE INVENTION

Accordingly, the present invention provides a method of treating a subject for prophylactic or therapeutic inflammatory conditions said method comprises administering effective amount of a composition comprising essentially a anti-inflammatory monodisperse macromolecular compound dendrimer or its analogues having a plurality of terminal groups and optionally said dendrimer or its analogues bound or complexed with drug moieties having an anti inflammatory activity or drug moieties assists in the anti-inflammatory activity to said subject.

In an embodiment of the invention, the subject is selected from mammals, human or animal and the inflammation is associated with arthritis, myositis, insect bites, sunburn, psoriasis, or atopic dermatitis, rheumatoid arthritis, multiple sclerosis, Guillain-Barre syndrome, Crohn's disease, ulcerative colitis, graft versus host disease, systemic lupus erythematosus, irritable bowel syndrome and insulin-dependent diabetes mellitus or inflammation associated with the pathophysiological condition of any disease i.e. alzhemier disease, asthama and soft tissue disease.

Still another embodiment the composition is administered practically by all routes namely parenteral, subcutaneous, intramuscular, intravenous, non-invasive routes selected form such as oral, mucosal, rectal, vaginal, intrauterinal, buccal, sublingual, nasal, ocular, ear, lung, transdermal and topical

Still another embodiment of the invention the said composition is administered as sterile or non-sterile formulation selected from solution, suspension, emulsion, elixirs, capsules, cachets, sachets, pills, tablets granules, powders, creams, solids, ointments, suppositories, lotions, film-forming solution, ointment, creams, gels, solutions, topical aerosols and pastes.

Yet another embodiment, dendrimer can also be used as pharmacologically acceptable drug delivery system selected from controlled drug delivery, sustained drug delivery, targeted drug delivery and intelligent drug delivery.

Yet another embodiment, the other delivery system for delivering the dendrimer alone or in combination with drug can be lipid based drug delivery systems, vesicular systems, nanoparticles, microspheres, microcapsules, cyclodextrins, calixarene, polymers and supramolecular biovectors.

Yet, another embodiment said dendrimer could also be used as an aqueous solubility enhancer for the drugs that assist in enhanced or synergistic activity.

Yet another embodiment solubility of dendrimer is can increased by electrostatic interaction, hydrogen bonding, chemical coupling, hydrophobic interaction, or physical inclusion of the said drug.

Yet another embodiment, the solubility enhancer property of dendrimer is mainly a subject of pH variation and type of generation used

Yet another embodiment, the said dendrimer is crosslinked at the surface or the entire network with biodegradable or non-biodegradable bonds, in which drugs are incorporated.

Yet another embodiment the said dendrimer can alter the biodisposition kinetics of the said drugs.

Yet another embodiment the amount of dendrimer used is in the range of 0.01 mg/kg to 1000 mg/kg as single or divided dose.

Yet another embodiment, the amount of drug used is in the range of 0.01 mg/kg to 1000 mg/kg

One more embodiment of the invention provides a method of treating an autoimmune disease, said method comprises administering to a subject in need of such treatment with a therapeutically effective amount of a composition comprising essentially a anti-inflammatory monodisperse macromolecular compound dendrimer or its analogues having a plurality of terminal groups and optionally said dendrimer or its analogues bound or complexed with drug moieties having an anti inflammatory activity or drug moieties assists in the anti-inflammatory activity to said subject.

Another embodiment of the invention, the autoimmune disease is selected from rheumatoid arthritis, acquired immuno deficiency syndrome, toxic shock syndrome, atherosclerosis, diabetes and inflammatory bowel disease.

One more embodiment of the invention provides an anti-inflammatory composition comprising of a monodisperse macromolecular compound dendrimer or its analogues having a plurality of terminal groups and/or such molecules attached (or distributed) to drug moieties having an anti-inflammatory activity or which assist in the anti-inflammatory activity.

Such compositions containing dendrimer referred in this invention is not only dendrimer but its pharmaceutically or veterinarily acceptable salts (alkaline metal or alkaline earth metal salts) and also its pharmaceutically or veterinarily acceptable analogues,

This invention also provides a pharmaceutical composition containing dendrimer, which not only have the inherent anti-inflammatory activity but also act as a macromolecular drug delivery system, which could be used for sustained and targeted delivery at the site of inflammation.

This invention also provides the complexes of the anti-inflammatory drugs with dendrimer, where dendrimer not only increases the solubility of the said drugs in water but also enhances the effect of the said drugs against the inflammation. Complexation of dendrimer with drug is mainly a subject of pH variation and type of generation used.

Drugs can either be encapsulated/entrapped inside the dendrimer or attached to the terminal groups of the dendrimer. Behavior of dendrimer and indomethacin in different pH values has been observed and optimum pH, where both dendrimer and indomethacin are optimally ionized was selected.

This invention further provides the composition for the treatment of inflammations, autoimmune disease and inflammation associated with the pathophysiological condition of any disease i.e. alzhemier disease, asthama, soft tissue disease etc. Anti-inflammatory activity of dendrimer was evaluated on rats in three different models—1. carrageenan foot edema test 2. cotton pellet test 3. adjuvant arthritis model. Dendrimer has shown the significant anti-inflammatory activity in all the three models.

Dendrimers are well-defined macromolecules that have a specific size, shape, and chemical functionality. The term ‘dendrimer’ is now used almost universally to describe highly branched monodisperse macromolecular compounds. Structurally they are highly branched macromolecules that can be subdivided into three architectural components: a central core branched cell, interior branch cell and branch cell possessing surface groups. They are synthesized through a stepwise repetitive reaction sequence. The present invention uses dendritic structures as frameworks for the attachment of ionic moieties; the invention is not limited to the spherical dendrimers described in detail herein but can be based on any dendritic structure. The variety of dendrimers in both shape and constitution are well known to persons skilled in the art.

The preparation of dendrimers is discussed in U.S. Pat. Nos. 4,507,466, 4,558,120, 4,568,737 and 4,587,329 (PAMAM dendrimers), as well as in U.S. Pat. Nos. 4,289,872 and 4,410,688 (lysine based dendrimer). Dendrimers has been reported to have antiviral activity in U.S. Pat. No. 6,190,650 and also used for antimicrobial treatment (U.S. Pat. No. 6,244,898). International Patent Publications Nos. WO 88/01178, WO 88/01179 and WO 88/01180 disclose conjugates or associates of dendrimer with another material such as a carried pharmaceutical or agricultural material. Nanoscopic sponges and nonogels of dendrimer were also formulated for the drug delivery (U.S. Pat. Nos. 5,938,934 and 6,333,051). Supramolecular property of dendrimer was utilized to encapsulate the molecule within a crosslinked shell molecule (U.S. Pat. No. 6,288,197). The term “dendrimer” as used in the present work is to be known in liberal way, which includes all the compositions, complexes, conjugates and formulations as discussed. The term also includes linked or bridged or crosslinked dendrimers as disclosed in the patents described previously. This term further includes any macromolecular compound, that is monodisperse and highly branched.

The preferred dendrimers of the present invention comprise a polyvalent core covalently bonded to at least two dendritic branches. Particularly preferred dendrimers are polyamidoamine (PAMAM) dendrimers, PAMAM (EDA) dendrimers, polylysine dendrimers, polypropylene dendrimer. The dendrimers of this invention may be prepared by standard chemical methods, which are well known, to persons skilled in this art. Biological evaluations of dendrimer show quite low toxicity.

The dendrimers of the present invention have been found to exhibit significant anti-inflammatory activity. As previously described, dendrimers are useful in prophylactic and therapeutic treatment of inflammation, for example osteoarthritis, multiple sclerosis, Guillain-Barre syndrome, Crohn's disease, ulcerative colitis, psoriasis, graft versus host disease, systemic lupus erythematosus, irritable bowel syndrome, insulin-dependent diabetes mellitus, rheumatoid arthritis, acquired immuno deficiency syndrome toxic shock syndrome, atherosclerosis, diabetes and inflammatory bowel disease or inflammation associated with the pathophysiological condition of any disease i.e. alzhemier disease, parkinsons disease, asthama, soft tissue disease etc.

Thus, in another aspect the present invention provides a pharmaceutical or veterinary composition for prophylactic or therapeutic anti-inflammatory treatment of a human or animal, which comprises a dendrimer as broadly described above optionally with a drug having anti inflammatory property or which assist in the anti-inflammatory property, in association with at least one pharmaceutically or veterinarily acceptable carrier or diluent or filler.

The formulation of such compositions is well known to persons skilled in this field. Suitable pharmaceutically acceptable carriers and/or diluents for parenteral and non-parenteral include any and all conventional solvents, dispersion media, fillers, solid carriers, aqueous solutions, coatings, antibacterial and antifungal agents, isotonic and absorption delaying agents, and the like. The proper utilization of above discussed agents with active substances is well know in the art.

The administration of the formulation is advisable to be given in discrete units containing therapeutically effective amount of the active substances with suitable diluents/fillers/carriers. The criterion for the chosen dosage unit depends upon the active substances, purpose and the type of dosage form used. In general, the compositions are prepared by uniformly and intimately bringing the active component into association with a liquid carrier, a finely divided solid carrier, or both, and then, if necessary, shaping the product.

In another aspect, the present invention provides a method for prophylactic or therapeutic treatment of inflammation in a human or non-human animal, which comprises administering to, said human or animal a prophylactic- or therapeutic-anti-inflammatory-effective amount of a dendrimer as broadly described above

In yet another aspect, this invention provides the use of a prophylactic- or therapeutic-anti-inflammatory-effective amount of a dendrimer as broadly described above in the prophylactic or therapeutic treatment of, or in the manufacture of a medicament for prophylactic or therapeutic treatment of an inflammation in a human or animal.

In general, route of administration can be any mode, which can produce, desired results without the unwanted side effect and should be medically acceptable. Such modes of administration include parenteral (e.g. subcutaneous, intramuscular and intravenous), oral, mucosal, rectal, vaginal, intrauterinal, sublingual, nasal, ocular, ear, lung, transdermal, topical. etc. Other routes include intrathecal administration directly into spinal fluid, direct introduction such as by various catheter and balloon angioplasty devices well known to those of ordinary skill in the art, and intraparenchymal injection into targeted areas.

The compositions may conveniently be presented in unit dosage form and may be prepared by any of the methods well known in the art of pharmacy. Such methods include the step of bringing the active component into association with a carrier that constitutes one or more accessory ingredients.

Compositions of the present invention suitable for oral administration may be presented as discrete units such as capsules, cachets, tablets granules, powders, lozenges, in liposomes or as a suspension in an aqueous liquor or non-aqueous liquid such as a syrup, an elixir, an emulsion, as a solution or as a gel, each containing a predetermined amount of the active component.

Compositions suitable for parenteral administration conveniently comprise a sterile aqueous preparation of the active component that is preferably isotonic with the blood of the recipient. This aqueous preparation may be formulated according to known methods using those suitable dispersing or wetting agents and suspending agents. The sterile injectable preparation may also be a sterile injectable solution or suspension in a non-toxic parentrally-acceptable diluent or solvent, for example as a solution in polyethylene glycol. Among the acceptable vehicles and solvents that may be employed are water, Ringer's solution and isotonic sodium chloride solution. In addition, sterile, fixed oils are conventionally employed as a solvent or suspending medium. For this purpose, any bland fixed oil may be employed including synthetic mono-or di-glycerides. In addition, fatty acids such as oleic acid find use in the preparation of injectables.

Compositions suitable for Topical administration conveniently comprises—ointment, creams, lotions, gels, solutions, topical aerosols and pastes—are composed of drug in suitable semisolid base which is either hydrophilic or hydrophobic in character. The topical base is selected from a wide variety of compositions, formulated according to known principals for pharmaceutical purposes. Such compositions include creams, solids, ointments, lotions, and film-forming solutions among others. They may be presented in boxes, jars, or compressible tubes, both collapsible and non-collapsible. The solids may be presented as sticks for rubbing onto the skin. Some of the topical bases may be presented as papers, woven or non-woven fabric pieces, or pads, all being impregnated with composition.

Composition of the present invention suitable for transdermal/topical administration may be presented as reservoir or microreservior type system which conveniently comprises of the active component as a solution or as a suspension. This may be formulated along with other known transdermal adjuvants, coenhancers or pH modifiers. Alternatively, a matrix type of this composition along with the active component and other transdermal polymers may be formulated.

Further delivery system can include targeted delivery systems. Targeted delivery system can be of two major types 1. Active targeting 2. Passive targeting. Dendrimer as a macromolecular drug delivery system can be targeted to the inflamed tissues by EPR (enhanced permeation and retention) effect. The term “sustained release” has been constantly used to describe a pharmaceutical dosage form formulated to retard the release of a therapeutic agent such that its appearance in the systemic circulation is delayed and/or prolonged and its plasma profile is sustained in duration. The onset of its pharmacological action is often delayed, and the duration of its therapeutic effect is sustained. Many types of sustained release delivery systems are available. These include, but are not limited to (a) erosional systems in which the active component is contained within a matrix, and (b) diffusional systems in which the active component permeates at a controlled rate through a polymer. In addition, a pump-based hardware delivery system can be used, some of which are adapted for implantation. Dendrimers were used to occlude the active substance with the help of blocking agents and the time and duration of release of active substance was controlled.

The term “controlled release”, on the other hand has a meaning that goes beyond the scope of sustained drug action. It also implies a predictability and reproducibility in the drug release kinetics, which means that the release of drug ingredients from a controlled release drug delivery system proceed at a rate profile that is not only predictable kinetically, but also reproducible from one unit to another. Rate controlled drug delivery systems can be classified as 1. Rate-programmed drug delivery system 2. Activation-modulated drug delivery system 3. Feedback-regulated drug delivery system 4. Site targeting drug delivery system.

Dendrimer is also used as solubility enhancers. However, there are some reports regarding this property of dendrimers but thorough evaluation was not performed. Solubility enhancement property is attributed to the ionizable groups in dendrimer which may be of primary, secondary or tertiary in nature, moreover dendrimer is a macromolecule and have a property of self-association, hence it will not behave as other solubility enhancers like cyclodextrin, calixarenes.

In solubility studies of PAMAM (fourth generation) with indomethacin, A_{N} type of solubility profile was observed, which meant negative deviation in the phase solubility diagram between dendrimer and indomethacin and was different from the previous studies reported using dendrimer. During initial pharmacodynamic studies of Dendrimer-indomethacin complex (DI) and indomethacin (I), it has been noticed that the effect of DI is not only sustained but found to be significantly more, indicating some additive/synergistic effect, which led us to study some anti-inflammatory activity of the dendrimer molecule itself.

pH has a prominent effect on the behavior of the dendrimer alone or in combination with the other drugs or delivery systems. Encapsulation or entrapment of the drugs in the dendrimer can be manipulated by the proper utilization of pH. Generation of dendrimer also have an important role to play in drug encapsulation/entrapment. Lower generation dendrimer behave in different manner than the higher generation dendrimer. Study of polyanionic dendrimer with positively charged nitroxide radicals suggest that increase in the size of the dendrimer leads to an increase in the size of hydrophobic cavity, favoring the encapsulation of more hydrophobic moieties.

The active component is administered in prophylactically or therapeutically effective amounts in single or multiple doses. Initially it starts with low dose and progressively doses may increase. Effective dose depends upon the route of administration, dosage form used, magnitude of the condition, age, weight etc. Dose can be increased or decreased from the range of therapeutic window, according to the condition. These factors are well known to those of ordinary skill in the art.

The objects, benefits, and advantages of our invention will become apparent from a consideration of the detailed description. The following examples are given by way of illustration and therefore should not be construed to the limit the scope of the present invention

### BRIEF DESCRIPTION OF THE ACCOMPANYING DRAWINGS

FIG. 1 represents structure of dendrimer

FIG. 2 represents Phase-Solubility diagram of Dendrimer and Indomethacin. (A_{N} type of curve.)

FIG. 3 represents the in-vitro sustained release study of indomethacin from the dendrimer formulation.

Formulations

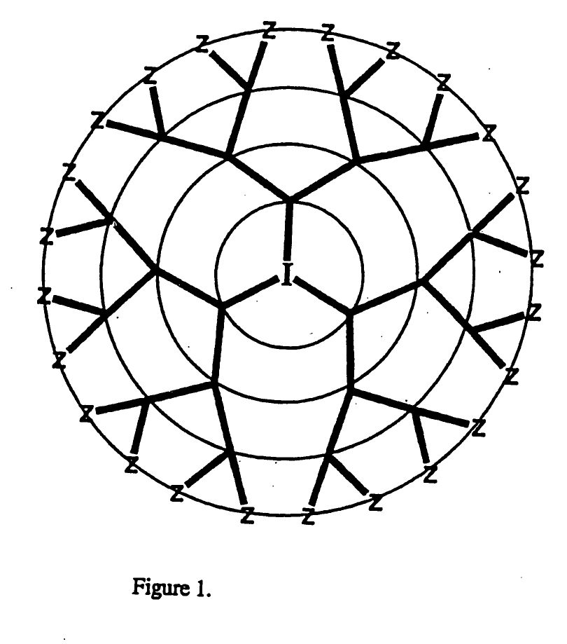

D=NH₂ terminated PAMAM dendrimer (EDA core) generation 4 (FIG. 1, Z=NH₂)
D2=aliphatic hydroxyl terminated PAMAM dendrimer (EDA core) generation 4 (FIG. 1, Z=OH)
I=Indomethacin 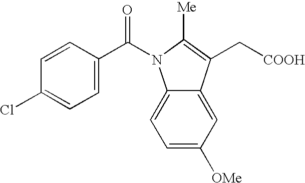
DI=Complex of indomethacin (I) and NH₂ terminated terminated PAMAM dendrimer (FIG. 1, Z=NH₂—Indomethacin)
D2-I=Complex of indomethacin (I) and aliphatic hydroxyl terminated PAMAM dendrimer (FIG. 1, Z=OH—Indomethacin)

### EXAMPLE-1

Materials. Dendrimers were purchased from Aldrich Chemical Co and were used as obtained.

### EXAMPLE-2

Sample preparation; Generation four, EDA core PAMAM dendrimer with —NH₂ terminal groups and generation four, EDA core PAMAM dendrimer with aliphatic OH-surface were used. Indomethacin containing PAMAM complexes were prepared by adding excess indomethacin powder in aqueous solutions of the dendrimers (pH-7), shaken in orbital shaker for 3 days at 300 rpm at 25° C., equilibrated, centrifuged at 800 g and then filtered with membrane filters (0.45 um, Whatman). Indomethacin was estimated by Ultraviolet spectroscopy (UV) at 320 nm.

### EXAMPLE-3

Phase Solubility Diagram

Solubility studies were carried out as described by Higuchi and Connors with minor modifications. The screw capped vial containing indomethacin (10 mg) in excess in aqueous dendrimer solutions (5.0 ml) at various concentrations (0.01% to 0.4%) and also at different pH, were shaken in orbital shaker at 25° C. for 3 days. After equilibration left at ambient temperature for 48 hrs and thereafter no further crystallization was observed, the solution was centrifuged at 800 g for 10 min, and supernatant was filtered through a membrane filter (0.45 um, whatman) and analyzed for indomethacin by UV 320 nm.

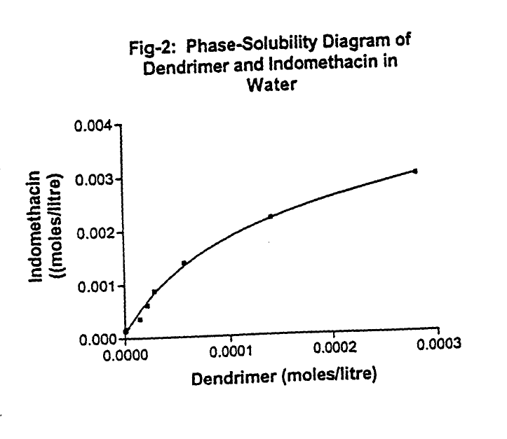

Solubility phase diagram depicts the A_{N} type of curve (FIG. 2), which represent a decreasing dependence on the ligand added at higher concentration. This type is comparatively least frequently encountered system, and its occurrence may be explained on the basis of self-association of the ligand at high concentration, which is in contrast to the curve reported by Emanuele et al, for dendrimer using ibuprofen. The negative slope in the curve may be due to the aggregation of dendrimer with the increasing concentration. Utility of dendrimer as a solubility enhancer is explained on the basis of electrostatic interaction of amino group of dendrimer with the carboxyl group of indomethacin. As the concentration of the ligand increases the dendrimer which has a supramolecular tendency, tends to aggregate thus decreasing the free amino groups available for the complexation and hence the solubility enhancement ability of the dendrimer. Expectedly, the aggregation of the dendrimer will be more with the increase in concentration, and hence the percentage of the available amino groups (in comparison with the theoretical amino group that should have been available in an ideal condition, i.e. in the absence of self aggregation) will decrease with increasing concentration.

### EXAMPLE-4

Comparative Behavior of Dendrimer and Indometacin at Different pH

To study the comparative behavior of dendrimer and indometacin at different pH two possibilities were tried. In case-I, three different concentration of dendrimer was added to the each of the aqueous solution with pH-2, 4, 7, 8 and 10 containing indomethacin. In Case-II, indomethacin was added to the each of the aqueous solution with pH-2, 4, 7, 8 and 10 of dendrimer at three different concentrations. The pH of the aqueous solutions of dendrimer was adjusted using 0.1 N HCl and 1M NaOH.

Case-I

Step-1

Indomethacin (in excess) was added separately to the aqueous solution of different pH (pH-2, 4, 7, 8 and 10) and kept for orbital shaking for 48 hrs. Then one half of the supernatant was filtered through a membrane filter (0.45 μm, Whatman) (case-A), and the remaining half along with the un-dissolved drug (i.e. in the suspension form) (case-B). Indomethacin solubilized was estimated by UV at 320 nm and pH was measured for both A and B.

Step-2

To A and B, three different concentration of dendrimer (0.075%, 0.15%, 0.2%) was added and agitated in an orbital shaker for 48 hrs. After equilibration was attained, the solution was centrifuged at 800 g for 10 min, and supernatant was filtered through a membrane filter (0.45 μm, Whatman) and analyzed for indomethacin by UV 320 nm and pH was measured in each case.

Case-II

Step-1

To the aqueous solutions of various pH (pH-2, 4 7, 8 and 10), dendrimer was added at different percentage. (0.075, 0.15, 0.2). pH was measured in each case.

Step-2

To all the above solutions (of step-1), indomethacin (in excess) was added and kept for orbital shaking for 48 hrs. After equilibration was attained, the solution was centrifuged at 800 g for 10 min, and supernatant was filtered through a membrane filter (0.45 μm, whatman) and analyzed for indomethacin by UV 320 nm and pH was measured in each case.

The study of the comparative behavior of indomethacin and dendrimer at different pH was undertaken as both the moeties are of different nature with respect to ionization and size and hence at different pH their behavior is a matter of critical evaluation.

### Case-B

Indomethacin is weak acid (pKa-4.5) and with increasing pH it is solubilized more in the aqueous solution as shown in case 1, step-1, B. Addition of dendrimer has further increased the solubility of indomethacin. Until the pH 8, indomethacin concentration increases with the increase in pH and also with the increase in dendrimer concentration. At pH 10, indomethacin concentration increases with the dendrimer concentration but was found to be less than the corresponding dendrimer concentration at pH 8. The amines of dendrimer will show maximum ionization at acidic pH and minimum ionization at basic pH. At pH 2 dendrimer will be fully ionized but indomethacin will be literally unionized, hence addition of dendrimer will not substantially increase the indomethacin concentration. At pH 4 to 8 (initial pH) addition of dendrimer has considerably increased the indomethacin concentration. At pH 10, the dendrimer will be practically unionized and hence the addition of dendrimer do not show much increase in indomethacin concentration. After pH 8 (initial pH) the indomethacin concentration starts decreasing, which is evident from the data of indomethacin concentration at pH 10. (initial pH). Critical comparison of the resultant pH of case-B with the pH of step-2 of case-2 (which is also its resultant pH), the pH range is found to be same, suggesting similar type phenomenon. In other words either add indomethacin in aqueous solution of dendrimer or vice-versa, the result and pattern would be same. The magnitude of increase in indomethacin concentration on addition of dendrimer was found to be more at pH 4, 7, and 8 since both dendrimer and indomethacin were optimally ionized at these pH values, but at pH 2 and 10 there is no significant increase in indomethacin concentration as one of the moiety is unionized in said pH and hence complexation could not take place.

### Case-A

A saturated solution of indomethacin obtained from filtering the aqueous solution of the drug was taken for the further study and dendrimer was added at different concentration. Incidentally, the solubility of the indomethacin was found to be decrease with the addition of dendrimer which may be due to high solubility of dendrimer in water. As the dendrimer has more preferentially solubility in water, it displaces indomethacin from the water and in this process, some indomethacin could have entered the crevices of the dendrimer.

High pH range of the resultant solution shows that dendrimer is predominantly present in the solution, and if we carefully compare the resultant pH in the case-A with the step-1 of case-2 (where only dendrimer is added to the different pH solutions), the pH range was found to be same, suggesting that in case-A, indomethacin is present in very less quantity and the most of dendrimer is in the free form and has not formed any complex with the solubilized indomethacin present in the solution as in case-B.

### EXAMPLE-5

Characterization of the DI Complex

¹H NMR Spectroscopy: ¹H NMR spectra were obtained with a Varian-Gemini spectroscopy. Samples were dissolved in deuterated methanol.

The evidence of Dendrimer-Indomethacin complex formation in aqueous solution was based on the modification of the ¹H-NMR spectrum of pure indomethacin, following the interaction between dendrimer and indomethacin. The ¹H-NMR data of indomethacin protons in the presence and absence of dendrimer are listed in Table-1. The only significant change was observed in the protons near to the carboxylic group, suggesting its involvement in the complex formation.

Infrared Spectroscopy: Infrared spectra of KBR discs of the samples were obtained using Perkin-Elmer 1420 infrared spectrometer.

The comparative infrared analysis indicate interaction between carboxyl functional group of indomethacin with amino group of dendrimer in formulation DI, (Table-2). The characteristic peak of —NH in dendrimer (D) has been shifted from 3250 cm-1 to 3264 cn-1 in DI. The absorption band of —NH—CO was deviated from 1550 cm-1 in D to 1536 cm-1 in DI. Peak due to —CH₂— has not shown significant deviation in DI compared to D and hence indicating the non-involvement of —CH₂— and involvement of —NH group and to the some extent of —CO—NH group of D in complex formation. The characteristic peak of carboxylic acid —H stretch (2954 cm-1), C═O stretch (1680 cm-1), carboxylic O—H out of plane deformation (952 cm-1) in indomethacin has found to be at 2928 cm-1, 1648 cm-1, 960 cm⁻¹ respectively in DI indicating involvement of these groups in complex formation. Band due to (C-0) stretch plus O—H deformation (1264 cm-1) and C—Cl (736 cm-1) showed no significant difference in indomethacin and DI, and hence their non-involvement in complex formation. Overall, result indicate the interaction between the carboxyl group of indomethacin and the —NH2 group of dendrimer.

Thermal Gravimetric Analysis: Thermal stability and degradation behavior were evaluated using Mettler Toledo thermogravimetric analyzer model between 50 and 1000° C. at heating rate of 20° C./min. The two step degradation behavior of DI in Thermogravimetrical analysis (TGA) further confirms the complex formation between dendrimer and Indomethacin.

### EXAMPLE-6

In-Vitro study: in-vitro release of indomethacin from dendrimeric formulation was performed using dialysis tube diffusion technique. A 1 ml aliquot of dendrimeric formulation was placed in the dialysis sac, hermetically tied and dropped into 40 ml of receptor medium containing phosphate buffer saline (PBS), pH7.4. The entire system was kept at 37° C. with continuous magnetic stirring. Samples of receptor solution were taken at various time intervals and assayed for indomethacin ultraviolet spectroscopy at 320 nm.

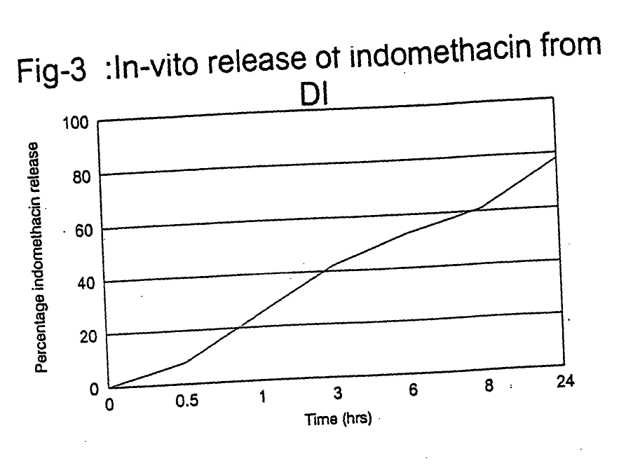

The in-vitro release study (FIG. 3) showed the sustained release of indomethacin from the dendrimer formulation. In 24 hrs DI complex has released 78% indomethacin. The delayed release of indomethacin from dendrimer could be attributed to the electrostatic interaction and/or to the hydrogen bonding between indomethacin and the dendrimer

### EXAMPLE-7

Evaluation for Anti Inflammatory Activity in In-Vivo Acute Model:

Carrageenan-induced edema in rats. Rats were dosed intraperitoneally with test formulations (D,I,DI) and saline. Five minutes later a subplantar injection of 0.1 ml of a 1% carrageenan was administered and volume of the injected paw was measured with water-displacement plethysmomerter, (UGO BASILE, ITALY), at one hour intervals for 8 hrs. The average paw swelling in a group of drug treated animals is compared with that of saline treated animals and the percentage inhibition of edema was determined. Results are recorded in Table 3 and Table-4

Acute study shows that the dendrimeric compounds (D, D2) are active and fulfill the requirement of the carrageenan induced model for inflammation as specified by Winter et al.

### EXAMPLE-8

Evaluation for Anti Inflammatory Activity in In Vivo Sub-Acute Model:

Cotton pellet test in rats: Four autoclaved pellets of cotton weighing 10 mg±0.5 mg were implanted on the previously shaved groin and axilla (two cotton pellet in each) region of rats aseptically. The test formulations (D,I and DI) were fed once a day from 1 to 7 of the experiment. On the day 8 th, the rats were etherized by an overdose of ether and the pellets surrounded by granuloma tissue wre dissected out carefully and dried in a hot oven at 60° C. till a constant weight was obtained. Percent inhibition compared to the control group was calculated and given in Table-5. Ulcers were not found in any of the case. After dissection various organs were removed, washed, dried and weighed and histopathology was carried out. No abnormalities were found after conducting the histopathology of liver, lung, kidney and spleen.

The subcutaneous implantation of cotton pellets provides inflammatory exudates that are easily processed. Implantation of cotton pellet triggers the series of cascade inflammatory reaction, leads to the migration of mediators of the inflammation in the zone of implantation. Dendrimeric formulations were found more effective than the standard indomethacin.

### EXAMPLE-9

Evaluation for Anti Inflammatory Activity in In Vivo Chronic Model:

Adjuvant-induced arthritis in rats: Complete Freunds adjuvant, Difco, (1 mg) was injected into the subplantar region of the right hind paw on day 0. Daily dosing with test drugs (D,I,DI) and saline begin one day prior to adjuvant injection (day-1) and terminated on day 15. At regular interval inflammatory response was measured by water-displacement plethysmometer, (UGO BASILE, ITALY). The average foot swelling in a group of drug treated animals is compared with that of saline treated animals and the percentage inhibition was determined. Results are recorded in Table 6. Data clearly indicate the superiority of dendrimeric formulations over the standard indomethacin. DI initially showed additive effect of D and I but on later stages effect of DI was either similar or less then the D, that is corroborating with the results of cotton pellet test. This may be due to the difference in mechanism of action or competitive inhibition of D and I and is matter of further evaluation.

Rats were sacrificed. After dissection various organs were removed, washed, dried and weighed. Ulcers were not observed in any of the cases. The histopathology studies revealed no abnormalities in liver, lung, kidney and spleen.

Hematological parameters were determined by blood cell counter (Medonic CA 620). The hematological pattern showed no significant difference between control and dendrimeric formulations (Table-7).

The body weight and feed intake of rats were monitored regularly. Weight gain has also been used as a parameter for assessing drug effectiveness. Compounds that are grossly toxic and therefore interfere with the rats' ability to exhibit an inflammatory response can be distinguished from those that are active, since therapy with the latter restores normal weight gain. Composition of this invention (D and DI) shows normal weight gain than I. Normal weight gain further supports that dendrimeric formulations are not toxic. Food consumption in the case of dendrimeric formulation (D and DI) was found to be slightly more than the saline and I, which may be due to the general debilitation associated with the inflammation.

TABLE 1
¹H-NMR chemical shift corresponding to indomethacin
in the presence and absence of dendrimer

| S. No | Indomethacin Proton | Δfree | δcomplex | δ |
|---|---|---|---|---|
| 1 | Aromatic A ring, 1H, | 6.6 | 6.65 | +0.05 |
| | multiplet | | | |
| 2 | Aromatic A ring, 1H, | 6.83 | 6.90 | +0.07 |
| | doublet | | | |
| 3 | Aromatic A ring, 1H, | 6.95 | 7.03 | +0.08 |
| | multiplet | | | |
| 4 | Aromatic C ring, 2H, | 7.5 | 7.55 | +0.05 |
| | doublet | | | |
| 5 | Aromatic A ring, 1H, | 7.63 | 7.7 | +0.07 |
| | doublet | | | |
| 6 | —CH3, 3H, singlet | 2.23 | 2.3 | +0.07 |
| 7 | —CH2, 2H, singlet | 3.63 | 3.5 | −0.13 |
| 8 | —OCH3, 3H, singlet | 3.8 | 3.8 | Nil |

δ = δ complex-δ free

TABLE 2
Comparative fourier transform infrared spectrum of D, I, and DI

| | Frequencies (cm−1) | | |
|---|---|---|---|
| Moieties | I | D | DI |
| —NH— | — | 3250 | 3264 |
| —NH—CO— | — | 1550 | 1536 |
| —CH2— | — | 1450 | 1456 |
| Carboxy acid O—H | 2954 | — | 2928 |
| stretch | | | |
| C═O stretch | 1680 | — | 1648 |
| (C—O) stretch plus O—H | 1264 | — | 1264 |
| deformation* | | | |
| Carboxylic O—H out of | 952 | — | 960 |
| plane deformation | | | |
| C—H out of plane | 900-600 | — | 900-600 |
| deformation for substituted | | | |
| aromatic | | | |
| Carboxylic O—H out of | 952 | | 960 |
| plane deformation | | | |
| C—Cl | 736 | — | 736 |
| Aromatic C═C stretch* | 1584 | — | 1584 |

TABLE 3
Mean percentage inhibition of D (15 mg/kg) in carrageenan
induced paw edema in rats. No of rats = 6

| | | Mean percentage |
|---|---|---|
| S. No. | Time (hrs) | inhibition ± SE |
| 1 | 1 | 46 ± 2.5 |
| 2 | 2 | 36 ± 1.9 |
| 3 | 3 | 32 ± 2.1 |
| 4 | 4 | 39 ± 2.2 |
| 5 | 5 | 39 ± 2.1 |
| 6 | 7 | 28 ± 1.1 |
| 7 | 8 | 29 ± 0.9 |

TABLE 4
Mean percentage inhibition Of D2 (15 mg/kg) in carrageenan
induced paw edema in rats. No of rats = 6

| | | Mean percentage |
|---|---|---|
| S. No. | Time (hrs) | inhibition ± SE |
| 1 | 1 | 38 ± 1.3 |
| 2 | 2 | 36 ± 1.6 |
| 3 | 3 | 36 ± 1.4 |
| 4 | 4 | 39 ± 2.1 |
| 5 | 5 | 28 ± 1.1 |
| 6 | 7 | 15 ± 0.8 |
| 7 | 8 | 13 ± 0.9 |

TABLE 5
Mean percentage inhibition in Cotton pellet test in rats.
No. of rats = 6

| | | | | Mean percentage |
|---|---|---|---|---|
| S no | Formulations | Dose | | inhibition ± SE |
| 1 | I | 1.4 | mg/kg | 22 ± 1.2 |
| 2 | D | 12 | mg/kg | 50 ± 3.1 |
| 3 | DI | 1.4 | mg/kgI | 47 ± 2.3 |
| | | 12 | mg/kg D | |

TABLE 6
Mean Percentage Inhibition in arthritic rats.
No. of Rats = 6

| Sl. | | | Mean Percentage Inhibition ± SE | | | | | | |
|---|---|---|---|---|---|---|---|---|---|
| No | Formulations | Dose | +1 day | +3 day | +5 day | +7 day | +10 day | +12 day | +14 day |
| 1 | I | 1 mg/kg | 17 ± 0.9 | 18 ± 0.7 | 9 ± 0.6 | 9 ± 0.8 | 8 ± 0.6 | 10 ± 0.9 | 11 ± 0.9 |
| 2 | D | 9 mg/kg | 24.5 ± 1.9 | 25 ± 2.1 | 30 ± 1.9 | 35 ± 2.4 | 29 ± 1.9 | 32 ± 2.4 | 30 ± 1.9 |
| 3 | DI | 1 mg/kg I | 35.16 ± 2.1 | 32 ± 2.9 | 35.4 ± 3.1 | 32 ± 2.1 | 21 ± 1.2 | 32 ± 1.8 | 27 ± 1.2 |
| | | 9 mg/kg D | | | | | | | |

TABLE 7
Study of hematological parameters in arthritic rats

| S. N. | PARAMETERS | SALINE | D | DI | I |
|---|---|---|---|---|---|
| 1 | Red blood cell (10⁶/mm³) | 6.93 | 6 | 6.56 | 5.87 |
| 2 | Mean cell volume for red blood cell(um³) | 50.9 | 49.7 | 52.1 | 52 |
| 3 | Red cell distribution width(%) | 14.2 | 14.9 | 13.5 | 18.9 |
| 4 | Red cell distribution width absolute (um³) | 36.9 | 36.3 | 37.2 | 44.1 |
| 5 | Haematocrit (%) | 35.2 | 37.3 | 36.6 | 30.3 |
| | Platelets(10³\mm³ | 613 | 700 | 592 | 778 |
| 6 | Mean Platelet Cell Volume (um³) | 6.58 | 6.5 | 6.25 | 6.55 |
| 7 | Platelet distribution width (um³) | 8.7 | 8.6 | 8.23 | 8.63 |
| 8 | Packed platelet volume (%) | 0.4 | 0.45 | 0.37 | 0.51 |
| 9 | Large platelets (%) | 8.45 | 7.75 | 6.5 | 8.05 |
| 10 | White blood cells (10³/mm³) | 7.58 | 8.78 | 7.33 | 8.85 |
| 11 | Hemoglobin concentration (gm/dl) | 13.2 | 11.3 | 12.4 | 11.6 |
| 12 | Mean cell hemoglobin(pg) | 19.1 | 18.9 | 19.5 | 19.9 |
| 13 | Mean cell hemoglobin concentration(g/dl) | 37.6 | 38 | 37.4 | 38.2 |
| 14 | Lymphocyte concentration (10³/mm³) | 3.4 | 3.6 | 3.73 | 3.43 |
| 15 | Granulocyte concentration (10³/mm³) | 3 | 3.4 | 2.73 | 4.73 |
| 16 | Mid-sized cells concentrtation (10³/mm³) | 0.65 | 0.6 | 0.75 | 0.7 |
| 17 | Lymphocyte concentration (%) | 48.6 | 46.7 | 55.2 | 39.9 |
| 18 | Granulocyte concentration (%) | 43.6 | 45.3 | 38.9 | 53.1 |
| 19 | Mid-sized cells concentrtation (%) | 7.85 | 7.95 | 7.16 | 7.05 |

### REFERENCES

Patent Document

1. Balogh, et al., Dendrimer-based nanoscopic sponges and metal composites, U.S. Pat. No. 5,938,934, Aug. 17, 1999
2. Denkewalter, et al., Macromolecular highly branched homogeneous compound based on lysine units, U.S. Pat. No. 4,289,872, Sep. 15, 1981.
3. Denkewalter, et al., Macromolecular highly branched homogeneous compound, U.S. Pat. No. 4,410,688, Oct. 18, 1983
4. Jansen et al., Composition containing of a dendrimer and a active substances European patent-0684044A2, 24 may, 1995.
5. Kabanov, et al. Nanogel networks and biological agent compositions thereof, U.S. Pat. No. 6,333,051, Dec. 25, 2001
6. Matthews, et al., Antiviral dendrimers, U.S. Pat. No. 6,190,650, Feb. 20, 2001
7. Suzuki, S, Waterproof connector, U.S. Pat. No. 6,244,898, Jun. 12, 2001
8. Tomalia, et al., Dense star polymer, U.S. Pat. No. 4,558,120, Dec. 10, 1985.
9. Tomalia, et al., Dense star polymers and dendrimers, U.S. Pat. No. 4,568,737, Feb. 4, 1986.
10. Tomalia, et al., Dense star polymers having core, core branches, terminal groups, U.S. Pat. No. 4,507,466, Mar. 26, 1985.
11. Tomalia, et al., Dense star polymers having two dimensional molecular diameter, U.S. Pat. No. 4,587,329, May 6, 1986.
12. Tomalia et al., starburst conjugates, WO88/01178., 25 Feb. 1988.
13. Tomalia et al., Bioactive and/or targeted dendrimer conjugates, WO88/01179., 25 Feb. 1988.
14. Tomalia et al., starburst conjugates, WO88/01180., 25 Feb. 1988
15. Tomalia et al., starburst conjugates, WO95/24221., 14 Sep. 1995
16. Youngs, et al., Supramolecular structures and process for making the same, U.S. Pat. No. 6,288,197, Sep. 11, 2001

### OTHER REFERENCES

17. Goodman and Gilman's The Pharmacological Basis of Therapeutics, pages 600-617, McGraw hill, New York, 1990.
18. Vane, J. R. and Botting, F. M., Inflamm. Res. 44:1-10, 1995.
19. Miyamoto, T., et al., Purification of prostaglandin endoperoxide synthase from bovine vesicular gland microsomes. J. Biol. Chem. 251:2629-2636, 1976.
20. Simmons, D. I., et al., Identification of phorbol ester-repressible v.s.c. inducible gene. Proc. Natl. Acad. Sci. USA 86:1178-1182, 1989
21. Masferrer, J. L., et al., Endogeneous glucocorticoid regulate an inducible cyclooxygenase enzyme. Proc. Natl. Acad. Sci. USA 89:3917-3921, 1992.
22. Lee, S. K., et al., Selective expression of mitogen-inducible cyclooxygenase in macrophages stimulated with lipopolysaccharides. J. Biol. Chem. 267:25934-25938, 1992
23. Meade, E. A., et al., Differential inhibition of prostaglandins endoperoxide synthase (cyclooxygenase) isozymes by aspirin and other non-steroidal anti-inflammatory drugs. J. Biol. Chem. 268:6610-6614, 1993
24. Mitchell, J. A., et al., Selectivity of non-steroidal anti-inflammatory drugs as inhibitors of constitutive and inducible cyclooxygenase. Proc. Natl. Acad. Sci. USA 90:11693-11697, 1994.
25. Taha, A., et al., N. Engl. J. Med. 334:1435-1439, 1996.
26. Stoddart, J. F. et al., Dendrimers-branching out from curiosities into new technologies, Prog. Polym. Sci., 23: 1-56 (1998)
27. Roberts, J. C. et al., J. Biomed. Mater. Res., 30:53-65 (1996)
28. Chien, Y. W., “Concept and system design for the rate-controlled drug delivery”, In Novel drug delivery systems, second edition, revised and expanded, Marcel Dekker Inc, New York 50:1-42 (1992)
29. Emanuele. A. D. et al. “Polyamidoamine starburst dendrimers as solubility enhancers” Int. J. Pharmaceutics, 197: 239-241 (2000)
30. Shinkai, S. et al., “Synthesis, metal-binding properties and polypeptide solubilization of crowned arborols” J. Chem. Soc. Perkin. Trans., 1:75-81 (1994)
31. Tomalta, D. A. and Durst, H. D., Top. Curr. Chem., 1993, 165, 193-313
32. Ottaviani, M. F., Wollley, K. L. and Frechet, J. M. J, Unimolecular micelles and globular amphiphiles: Dendritic macromolecules as a novel recyclable solubilization agent, J chem. soc. Perkin Trans. 1, 1993, 1287-1297.
33. Highchi, T., and Connars, K. A., Advances in analytical chemistry and instrumentation, vol. 4 (C. N. reilly, ed.), Interscience, New york, 1965, P117
34. Bliven, M. L., and Otterness, I, G., “Laboratory models for testing NSAIDS' in Nonsteroidal Antiinflammatory Drugs, A Wiley Interscience Publication, New york, 1985 pp (172)
35. Winter, C. E. et al., Carrageenan induced edema in hind paw of the rat as an assay for anti-inflammatory drugs. Proc. Soc. Exp. Bio. Med., 111: 544 (1962)

1. A composition for treating inflammation diseases or pathological conditions involving inflammation, the said composition essentially comprises a anti-inflammatory monodisperse macromolecular compound dendrimer or its analogues having a plurality of terminal groups and optionally such molecules bound or complexed with drug moieties having an anti inflammatory activity or drug moieties which assist in the anti-inflammatory activity.

2. The composition according to claim 1, wherein the anti-inflammatory macromolecule is a monodisperse dendrimer or its analogues, having a property of encapsulating or capable of forming a covalent or non-covalent complex with anti-inflammatory drug molecules or drug moieties which assist in the anti-inflammatory activity.

3. The composition according to claim 1, wherein the said dendrimer comprises of polyvalent core covalently bonded to at least two dendritic branches.

4. The composition according to claim 1, wherein the said dendrimer is selected from the group consisting of polyamidoamine dendrimer, polypropylene dendrimer, polyethyleneimine dendrimer, carbohydrate based dendrimer, peptide based dendrimer, glycopeptide dendrimer, metal containing dendrimer, poly aryl amine dendrimer, polyamide dendrimer, poly (alkyl amine) dendrimer, polyamido alcohol dendrimer, cyano dendrimer, polyether dendrimer, polythioether dendrimer, polysiloxane dendrimer, dendritic aryl ester, perchlorinated dendrimer, catylitic centre containing dendrimer, silicon containing dendrimer, phosphorus containing dendrimer, hydrocarbon dendrimer, or any molecule possessing dendritic framework of controlled architecture.

5. The composition according to claim 1, wherein the dendrimer is constructed of analogues of drug molecules possessing anti-inflammatory activity.

6. The composition according to claim 3, wherein said polyvalent core is selected from group consisting of ammonia, alkylenediamine, peptide, aryl, pentaerythritol, metallocores, porphyrins, polyalkylsilane or any such molecule on which the dendrimer can be synthesized.

7. The composition according to claim 1 wherein the terminal group is anionic or cationic in nature.

8. The composition according to claim 1, wherein the said dendrimers has terminal groups selected from the group consisting amino, hydroxyl, carboxylate, thiol, boronic acid, metal chelates, cyano or any such functional terminal groups which are sufficiently reactive or capable of having covalent or non-covalent interaction.

9. The composition according to claim 1, wherein the dendrimer is a generation 0 to generation 10 poly(amidoamine) dendrimer, or a generation 0 to generation 5 poly(propyleneimine) dendrimer.

10. The composition according to claim 1, wherein the dendrimer used has low toxicity.

11. The composition according to claim 9, wherein the surface groups of dendrimer ranges from about 3 to about 4100

12. The composition according to claim 9, wherein the molecular weight of the dendrimer ranges from about 350 to about 935,000

13. The composition according to claim 9 in which the molecular diameter of the dendrimer ranges from about 5 to about 200 Angstrom

14. The composition according to claim 1, wherein said drug has anti-inflammatory activity of its own or which assist in the anti-inflammatory activity.

15. The composition according to claim 1, wherein the said drug is selected from the group consisting of cyclooxygenase inhibitors, non-steroidal antiinflammatory drugs (NSAIDs), antigout drugs, anti-rheumatoid drugs, 5-lipooxygenase inhibitors, cysteinyl leukotriene receptor antagonist, cytokines inhibitors, phosphodiesterase inhibitors, H₁ receptor antagonist, immunomodulators, immunosuppressive agents or any such molecule which has potential anti-inflammatory activity or assist in the same.

16. The composition according to claim 1, wherein the said drug is used alone or in combination with other anti-inflammatory drugs or which assist in anti-inflammatory activity

17. The composition according to claim 1, wherein the anti-inflammatory drugs are Non-steroidal anti-inflammatory drugs (NSAIDs) selected from the group consisting of anthranilic acids, acetofenac, amfenac, aclofenac, aspirin (5-acetylsalicylic acid), azodisal sodium, benoxaprofen, bromofenac clidanac, celecoxib, carboheterocyclic acids, carprofen, chlorambucil, diclofenac, difimsial, etodolac, ensfenamic acid, etodolic acid fenbufen, fenclofenac, fenclorac, fenclozic acid, fenoprofen, flufenamic acid, flurbiprofen, fluprofen, furosemide, gold sodium thiomalate, ibuprofen, indomethacin, indoprofen, isofezolac, ketorlac, ketoprofen, lonazolac, loxoprofen, meclofenamic acid, mefanamic acid, meclofenamate, melphalan, oxaprozin, naproxen, nimuselide, niflumic acid, penicillamin, phenylacetic acids, pirprofen, pranoprofen, proprionic acids, refecoxib salicylic acids, salazosulfapyridine, sulindac, tolmetin, a pyrazolone butazone propazone, meloxicam, oxicams, piroxicam, feldene, piroxicam beta cyclodextran, suprofen, tolmetin, tolfenamic acid, tenoxicam, zamopirac and zaltoprofen.

18. The composition according to claim 15, wherein the said anti-rheumatoid drugs are selected from the group consisting of gold compound, pencillamine, sulphasalazine, methotrexate, chloroquine, hydroxychloroquine, azathioprene, cyclosporine, glucocorticoids and leflunomide

19. The composition according to claim 15, wherein the said anti-gout drugs are selected from the group consisting of allopurinol, probenecid, sulphinpyrazone and colchicines.

20. The composition according to claim 15, wherein the said H₁ receptor antagonist are selected from the group consisting of diphenhydramine, promethazine, chlorpheniramine, mequitazine, astemizole, cyclzine, dimenhydrinate, cinnarizine, mepyramine, mequitazine, terfenadine, fexofenadine, loratidine, cetrizine and cyproheptadine.

21. The composition according to claim 15, wherein the said immunosuppressive agents are selected from the group consisting of cyclosporine, tacrolimus, rapamycin, glucocorticoids, corticosteriods, cyclophosphamide, chlorambucil, azathioprine, myclophenolate, mofetil, immunoglobulins and rapamycin

22. The composition according to claim 1 wherein the said composition is a complex with which said drug is covalently or non-covalently attached, entrapped, encapsulated or occluded to dendrimer by physical or by chemical bonding.

23. The composition according to claim 22, wherein the said drug is interacted to the primary terminal groups or internal secondary/tertiary groups of dendrimer with covalent and non-covalent interactions.

24. The composition according to claim 23, wherein the said interaction is mainly dependent on pH variation and type of generation used

25. The composition according to claim 1, wherein the said dendrimer can form supramolecular structure with itself or with other molecules or drugs.

26. The composition according to claim 25 wherein the said supramolecular dendrimer can be used for the delivery of drugs or therapeutically active substances

27. The composition according to claim 1, wherein the said dendrimer is attached to the ligand specific for cell type, which is taken up by cell surface receptors with or without internalization

28. The composition according to claim 27, wherein the said ligand is complexed with dendrimer or its analogues by covalent or non-covalent interaction with or without biodegradable bonds.

29. The composition according to claim 27 wherein the targeting ligand is attached to the dendrimer constructed of anti-inflammatory molecules

30. The composition according to claim 1, wherein the pathological conditions selected form the inflammation associated with arthritis, myositis, insect bites, sunburn, psoriasis, or atopic dermatitis, rheumatoid arthritis, multiple sclerosis, Guillain-Barre syndrome, Crohn's disease, ulcerative colitis, graft versus host disease, systemic lupus erythematosus, irritable bowel syndrome and insulin-dependent diabetes mellitus or inflammation associated with the pathophysiological condition of any disease i.e. alzhemier disease, parkinsons disease, heart disease, asthama and soft tissue disease.

31. A pharmaceutical or veterinary composition for prophylactic or therapeutic anti-inflammatory treatment of human or mammal, said composition essentially comprises a anti-inflammatory monodisperse macromolecular compound dendrimer or its analogues having a plurality of terminal groups, optionally such dendrimer or its analogues bound or complexed with drug moieties having an anti inflammatory activity or drug moieties assists in the anti-inflammatory activity and in association with at least one or more pharmaceutically or veterinarily acceptable carrier or diluent.

32. A method of treating a subject for prophylactic or therapeutic inflammatory conditions said method comprises administering effective amount of a composition comprising essentially a anti-inflammatory monodisperse macromolecular compound dendrimer or its analogues having a plurality of terminal groups and optionally said dendrimer or its analogues bound or complexed with drug moieties having an anti inflammatory activity or drug moieties assists in the anti-inflammatory activity to said subject.

33. The method as claimed in claim 32, wherein the subject is selected from human or animal.

34. The method as claimed in claim 32, wherein the inflammation is associated with arthritis, myositis, insect bites, sunburn, psoriasis, or atopic dermatitis, rheumatoid arthritis, multiple sclerosis, Guillain-Barre syndrome, Crohn's disease, ulcerative colitis, graft versus host disease, systemic lupus erythematosus, irritable bowel syndrome and insulin-dependent diabetes mellitus or inflammation associated with the pathophysiological condition of any disease i.e. alzhemier disease, asthama and soft tissue disease.

35. A method as claimed in claim 32, wherein the said composition is administered practically by all routes namely parenteral, subcutaneous, intramuscular, intravenous, non-invasive routes selected form such as oral, mucosal, rectal, vaginal, intrauterinal, buccal, sublingual, nasal, ocular, ear, lung, transdermal and topical

36. A method as claimed in claim 32, wherein the said composition is administered as sterile or non-sterile formulation selected from solution, suspension, emulsion, elixirs, capsules, cachets, sachets, pills, tablets granules, powders, creams, solids, ointments, suppositories, lotions, film-forming solution, ointment, creams, gels, solutions, topical aerosols and pastes.

37. A method as claimed in claim 32, wherein dendrimer can also be used as pharmacologically acceptable drug delivery system selected from controlled drug delivery, sustained drug delivery, targeted drug delivery and intelligent drug delivery.

38. A method as claimed in claim 32 wherein, dendrimer alone or in combination with drug can be used with other drug delivery system i.e. lipid based drug delivery systems, vesicular systems, nanoparticles, microspheres, microcapsules, cyclodextrins, calixarene, polymers and supramolecular biovectors.

39. A method as claimed in claim 32, wherein dendrimer can also be used as an aqueous solubility enhancer for the drugs that assist in enhanced or synergistic activity.

40. A method as claimed in claim 39, wherein dendrimer can increase solubility by electrostatic interaction, hydrogen bonding, chemical coupling, hydrophobic interaction, or physical inclusion of the said drug.

41. A method as claimed in claim 39, wherein the said solubility enhancer property of dendrimer is mainly a subject of pH variation and type of generation used

42. A method as claimed in claim 32, wherein the said dendrimer is crosslinked at the surface or the entire network with biodegradable or non-biodegradable bonds, in which drugs are incorporated.

43. A method as claimed in claim 42, wherein dendrimer can alter the biodisposition kinetics of the said drugs.

44. A method as claimed in claim 32, wherein dose of dendrimer is in the range of 0.01 mg/kg to 1000 mg/kg as single or divided dose.

45. A method as claimed in claim 32, wherein dose of the drug is in the range of 0.01 mg/kg to 1000 mg/kg

46. A method of treating an autoimmune disease which comprises administering to a subject in need of such treatment with a therapeutically effective amount of a composition comprising essentially a anti-inflammatory monodisperse macromolecular compound dendrimer or its analogues having a plurality of terminal groups and optionally said dendrimer or its analogues bound or complexed with drug moieties having an anti inflammatory activity or drug moieties assists in the anti-inflammatory activity to said subject.

47. A method as claimed in claim 46, wherein the autoimmune disease is selected from rheumatoid arthritis, acquired immuno deficiency syndrome, toxic shock syndrome, atherosclerosis, diabetes and inflammatory bowel disease.
